(19) United States
(12) Patent Application Publication
(10) Pub. No.: US 2003/0156974 A1
(43) Pub. Date: Aug. 21, 2003

(54) Mold prevention system and methods

(76) Inventors: Douglas A. Haas, Dickinson, TX (US); Timothy L. Allen, Friendswood, TX (US)

Correspondence Address: MICHAEL E. MARTIN, GARDERE WYNNE SEWELL LLP, SUITE 3000, 1601 ELM STREET, DALLAS, TX, 75201, US

(21) Appl. No.: 10/078,917

(22) Filed: Feb. 20, 2002

(51) Int. Cl. A61L 2/00

(52) U.S. Cl. 422/28; 422/33; 422/292

(57) ABSTRACT

Growth of toxic molds in residential and commercial buildings is prevented or eradicated by installing a system including liquid spray nozzle units within interior walls spaces likely to accumulate moisture. Spray nozzles are mounted in spaces between column or stud members of interior walls, plumbing chases, or similar locations and may be manifolded and connected to a fitting projecting through a wall in an inconspicuous location for connection to a portable pump unit for spraying surfaces of structural elements adjacent the spray nozzles. Methods for treating building interiors include applying a spray of anti-microbial treatment fluid to interior facing surfaces of exterior walls, plumbing chases, interior bathroom and kitchen wall column members and floor plates, floors, such as concrete slabs or wood flooring surfaces, roof penetrations and subfloors for utility appliances.

### BACKGROUND

Growth of fungi, such as certain types of black or gray molds in residential and commercial buildings, can produce significant health hazards to human occupants of such buildings. For example, the black molds known as Stachybotrys and Memnoniella are types that are known to produce mycotoxins which are hazardous to human health when exposure is encountered. These mold types typically occur on repeatedly wetted materials that contain cellulose, for example, including interior wall paneling, such as gypsumboard, and other materials used in residential and commercial buildings including cardboard, ceiling tiles, cellulose insulation, wood, etc.

There are several sources of moisture which can accelerate and promote the growth of gray and black molds including the types mentioned above. Water piping extending through plumbing chases and piping extending through voids or spaces in interior walls of residential and commercial buildings can produce minute leaks sufficient to wet adjacent materials and promote the growth of mold. Leakage from the exterior of a building into various parts of the building, which goes undetected, can also wet the surfaces of various materials which will promote the growth of toxic molds. Still further, condensation, from time to time, on the surfaces of plumbing piping, air conditioning ducts, refrigerant conduits and other structures is also a source of moisture which can result in the growth of molds in unseen spaces, such as the interior wall spaces of buildings, among other locations, all of which are a source of mold contamination and exposure to humans.

The growth of mold in interior wall spaces in residential and commercial buildings is particularly difficult to detect and difficult to eradicate by conventional methods. Since the interior wall spaces have been covered with various types of wall paneling, the growth of mold goes undetected and, when detected, is difficult to treat without major renovation of the building. Accordingly, there has been a longstanding need for the development of mold and other pest prevention systems and treatment methods which can eliminate toxic molds, in particular, from various places within a residential or commercial building, including interior wall spaces, plumbing chases, and virtually any portion of a building structural feature which is likely to be exposed to moisture, repeatedly, during the life of the building. It is to these ends that the system and methods of the present invention have been developed.

### SUMMARY OF THE INVENTION

The present invention provides a system and methods for preventing or eliminating fungi, such as gray or black mold, from various surfaces of a residential and/or commercial building including interior wall spaces, plumbing chases and other portions of a building structure which are likely to serve as a place for growth of such mold.

In accordance with one aspect of the present invention, a mold prevention or eradication method is provided which includes the steps of removing all construction debris interfering with the application of a chemical mold treatment substance, such as an anti-microbial liquid, during construction, remodeling or repair of a building interior. The method includes spray application of a suitable anti-microbial fluid by delivery of a spray of a predetermined liquid particle size to surfaces which are likely to allow growth of mold thereon. A method in accordance with the invention may include that of applying the anti-microbial treatment fluid to all exposed interior surfaces of exterior walls of the building or structure, which may be done prior to installing interior wall panels or coverings at said exterior walls. The method also may include applying the treatment fluid, such as an anti-microbial liquid, to all plumbing chases and interior walls in rooms which include plumbing piping, such as bathrooms, kitchens, and the like. The treatment method preferably further includes applying the treatment fluid to wall column members, or so-called studs, floor plates, the interior surfaces of wallboard or paneling and other structural features which include cellulose or other mold growth promotion materials as part of the composition of the structural member. Still further, the treatment method involves application of a spray of the treatment fluid to the perimeter of the building flooring, a predetermined distance in from an exterior wall, including treatment of second and third story subflooring and decking, and treatment at or in the vicinity of all roof penetrations of the building.

A method in accordance with the invention also may be carried out by a system in accordance with the invention as will be explained in detail herein.

In accordance with another aspect of the present invention, a mold prevention and treatment system is provided which may be installed in various locations which are susceptible to the growth or harboring of toxic molds and vermin, including the spaces between interior wall panels, which spaces include plumbing or other sources of water leakage or condensation. The mold prevention and treatment system preferably includes a single or multi orifice liquid spray delivery system which is preinstalled and supported between opposed panels of an interior wall structure, for example. The delivery system also preferably includes a coupling or connector disposed in a location normally hidden from view so that, from time to time, a source of pressurized anti-microbial treatment fluid may be connected to the delivery system for spraying the spaces which are susceptible to mold growth or other contamination.

The system may be installed in new construction or during major repair or renovation of a structure at which the eradication of mold is desired or has been required. Moreover, the system comprises one or more spray nozzles which are operable to emit a fine mist of an anti-microbial treatment fluid, preferably in liquid form, in the vicinity of likely locations for mold growth to prevent or eliminate such growth. The system provides access to likely locations of mold growth or other contamination which are hidden from view by the occupants of the building and are normally inaccessible unless major structural modifications are made to the building.

Those skilled in the art will further appreciate the advantages and superior features of the system and methods of the present invention together with other important aspects thereof upon reading the detailed description which follows in conjunction with the drawings.

### BRIEF DESCRIPTION OF THE SEVERAL VIEWS OF THE DRAWINGS

FIG. 1 is a perspective view of a residential dwelling unit or house, with the roof structure removed, to illustrate typical locations for a prevention and treatment system in accordance with the invention;

FIG. 2 is a detail elevation view taken generally from the line 2-2 of FIG. 1;

FIG. 3 is a view taken substantially from the line 3-3 of FIG. 2; and

FIG. 4 is a view taken generally from the line 4-4 of FIG. 1.

### DETAILED DESCRIPTION OF THE PREFERRED EMBODIMENTS

In the description which follows, like elements are marked throughout the specification and drawing with the same reference numerals, respectively. The drawing figures are not necessarily to scale and some elements may be shown in somewhat generalized or schematic form in the interest of clarity and conciseness.

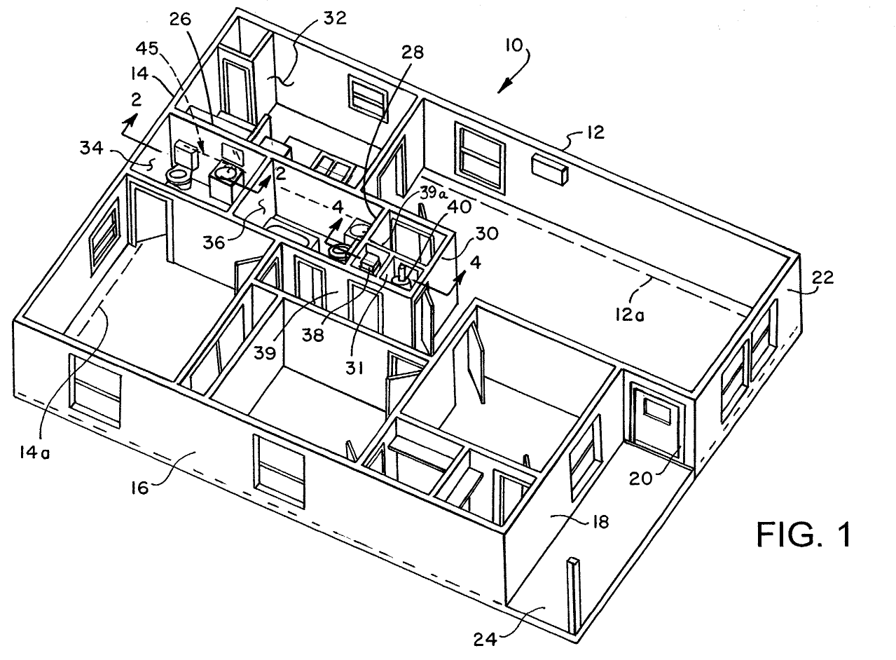

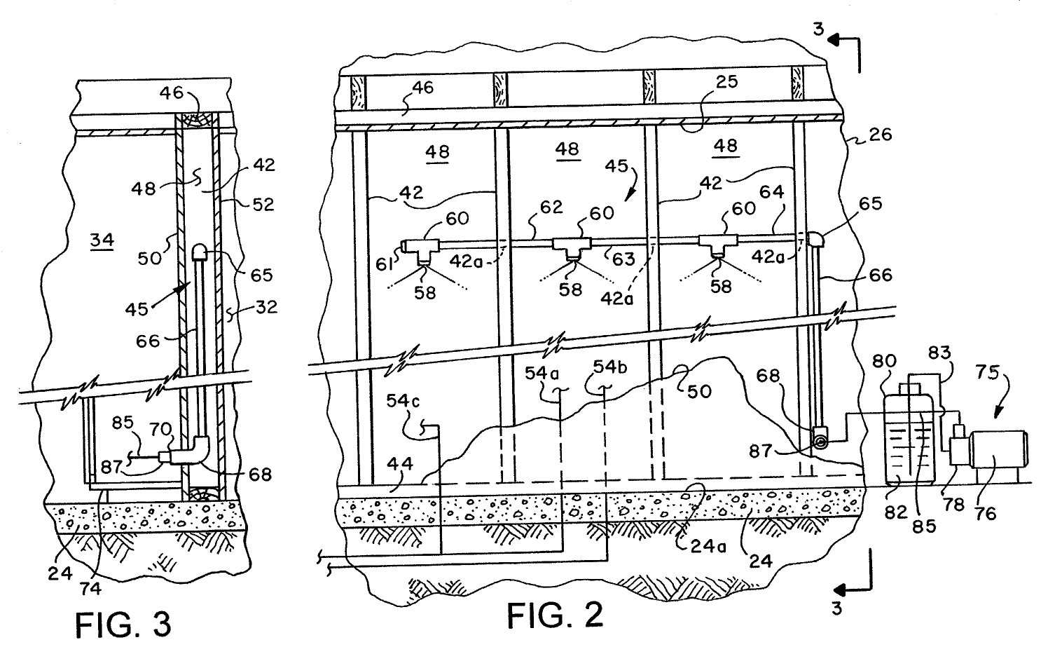

The mold prevention and treatment system and methods of the invention may be carried out in conjunction with many types of residential dwelling units, commercial buildings, or virtually any structure which may be occupied by humans and which may be susceptible to contamination, including the growth of mold of the types discussed herein. By way of example only, FIG. 1 illustrates, in perspective view, the interior of a residential dwelling unit or house 10 which includes exterior walls 12, 14, 16, 18, 20, and 22 enclosing a dwelling unit having several rooms, as illustrated. The dwelling unit 10 may be built on a concrete slab type foundation 24 or one of many other types of foundations commonly used for residential or commercial structures. The interior of dwelling unit 10 is subdivided by plural interior walls, certain ones of which are identified herein by numerals 26, 28, 30, 31, 39 and 39a by way of example only, and which contribute to dividing the interior of the dwelling unit into plural rooms such as a kitchen 32, first and second bathrooms 34 and 36, an air conditioning unit closet 38 and a water heater closet 40, for example, see FIGS. 2 and 3, also. Each of the aforementioned rooms has plumbing piping leading thereto and disposed within certain interior walls, such as the wall 26 for example. Each of the piping locations within interior walls may be subject to leakage or condensation on the exterior surfaces of the piping and which can wet the surfaces of the adjacent walls and/or floors.

Referring to FIGS. 2 and 3, by way of example, there is illustrated a portion of interior wall 26 extending between a floor surface 24a and a ceiling structure including paneling 25. Interior wall 26 is of somewhat conventional construction including spaced apart vertical column members 42, commonly known as studs, which are typically formed of wood, such as pine. Column members or studs 42 are secured to an elongated wood floor plate member 44 and to a ceiling plate or joist member 46 and form plural spaces 48 therebetween and between wall paneling members 50 and 52. In FIG. 2 a portion of wall panel 50 is broken away to show the spaces 48 separated by the column members 42. Since interior wall 26 forms one wall of bathroom 34, for example, there exist certain pressure water conduits 54a, 54b, and 54c extending therewithin and leading to certain fixtures, not shown, within bathroom 34. Additionally, certain kitchen fixtures, not shown, may include one or more water conduits or lines leading thereto, which water lines may also extend within one or more of the spaces 48.

The spaces 48 are susceptible to moisture which may originate from a minute leak in one or more of the pressurized water lines 54a, 54b, or 54c. Moisture may also condense on one or more of these water lines if the condition of ambient atmospheric air within the dwelling unit 10 and the temperature of the water flowing through one or more of the water lines is conducive to such condensation. The materials of which the wall paneling 50 and 52 and the structural members 42 and 44 are formed often contain cellulose on which certain types of mold, as described previously herein, will grow profusely.

A method in accordance with the present invention for treatment of the building or dwelling unit 10 may be carried out during or upon substantial completion of construction or major remodeling of the building. Such method will be described in further detail hereinbelow. However, one aspect of the invention provides a system for mold eradication and prevention, which system may also be installed during new construction or major remodeling of the building 10, for example.

At least one part of a mold prevention system of the invention is illustrated by way of example in FIGS. 2 and 3, and is generally designated by numeral 45. Mold prevention system 45 includes one or more spray nozzles 58 formed integral with or suitably secured to T-type fittings 60 which are manifolded together by a set of conduit sections 62, 63 and 64, for example. The manifold formed by the conduit sections 62, 63 and 64 and the fittings 60 may be installed during new construction of the dwelling unit 10 or during remodeling or renovation. The manifolded spray nozzles 58 are connected at one end of conduit 64 to a further conduit section 66 via an elbow fitting 65 extending to a fitting 68 which may have a connector end 70 extending through wall panel 50 and disposed in a hidden location, such as within a cabinet or enclosure 74, FIG. 3. The endmost fitting 60 is closed by a suitable plug 61, FIG. 2.

The system 45, comprising the manifolded spray nozzle arrangement illustrated in FIGS. 2 and 3, may be connected, from time to time, to a source of fungicide including a portable electric motor driven pump unit generally designated by the numeral 75 in FIG. 2. The pump unit 75 includes a suitable electric motor 76 drivingly connected to a centrifugal, high pressure pump 78 operable to deliver liquid fungicide to the manifolded spray nozzles 58 at a pressure of up to about 200 psig. The pump unit 75 is adapted to be connected to a source of fungicide including a portable reservoir 80 having a quantity of liquid pesticide or fungicide 82 therein and which may be of a type commercially available and known to be suitable for a eradicating mold of the types described herein or other vermin which may require eradication from time to time. Flexible conduits 83 and 85, FIG. 2, interconnect pump 78 with reservoir 80 and pump 78 with a suitable quick disconnect fitting 87 which may be releasably connected to fitting 68 at connector end 70.

Accordingly, the mold prevention and treatment system 45 shown in FIGS. 2 and 3 or a modified form thereof may be installed in any one of the interior walls of the dwelling unit 10 which include water piping, or are likely to be exposed to moisture. In this way, the dwelling unit 10 may be treated, from time to time, on a maintenance basis to eradicate or prevent the accumulation of toxic molds within the wall spaces enclosed by the interior walls, including the interior walls 26 and 28.

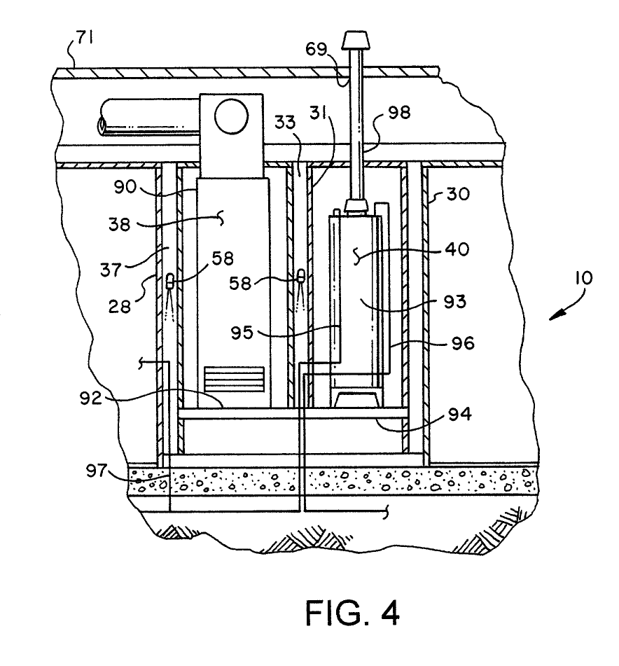

For example, referring briefly to FIG. 4, an interior wall 31 separates the air conditioning unit closet 38 from the water heater closet 40. As shown in FIG. 4, the closet 38 includes a conventional forced air heating and air conditioning unit 90 mounted on a suitable subfloor 92 and enclosed by walls 28 and 31, as well as interior wall 39 and an interior wall 39a, FIG. 1. In like manner, water heater closet 40 is adapted to be provided with a conventional water heater 93 mounted on a subfloor 94, which may be an extension of subfloor 92, as shown. Pressure water lines 95 and 96 extend partly within a space 33 within interior wall 31. Space 33 also includes one or more spray nozzles 58 mounted therein and operably connected to a fitting, such as the fitting 68, not shown in FIG. 4. Still further, one or more manifolded spray nozzles 58 are disposed in interior wall space 37 formed within interior wall 28, also as shown in FIG. 4, for discharging fungicide into the space 37 to prevent or eradicate mold which might grow within the space as a consequence of leakage from or condensation on pressure water line 97.

It will be noted that FIG. 4 also illustrates a penetration 69 through a roof deck 71 for a vent pipe 98 for water heater 93 which should also be treated for mold prevention or eradication in accordance with a method of the present invention.

Accordingly, with various single or plural manifolded spray nozzles 58 disposed within interior walls of the dwelling unit 10 and connected to suitable fittings 68 at various hidden locations throughout the dwelling unit, as illustrated by way of example in FIGS. 2 and 3, the portable pump unit 75 and reservoir 80 may be carried from one location to another within the dwelling unit, periodically, suitably connected to respective fittings 68 and energized so that a quantity of mold eradicating anti-microbial fluid, preferably in liquid form, may be discharged into the spaces likely to provide for growth of mold, for example. In this way, the dwelling unit 10 may be prevented from acquiring growth of toxic mold or the like. Those skilled in the art will also recognize that the system and methods of the invention may be carried out using other anti-microbial treatment fluids, including fungicide or pesticide-type fluids, to rid or eradicate contaminations of various types other than molds of the types described herein.

The installation of the nozzles 58, their associated fittings 60 and 68 and connecting piping may be carried out during construction of a building, such as the dwelling 10, or during major remodeling or, as required after initial construction has been completed.

One method in accordance with the present invention contemplates that mold prevention and eradication within a building such as the dwelling unit 10, may be carried out as described hereinbelow.

During construction of a building, such as the dwelling unit 10, a requisite number of spray nozzles 58 and associated fittings and conduits are installed, generally as described above and as shown in the drawing figures. A typical spray nozzle or misting nozzle 58 may be of a type commercially available, have a 0.008 inch diameter orifice mounted in a stainless steel T-fitting 60 with 0.375 inch internally threaded national pipe thread construction. The fittings 60 may be connected to 0.375 inch diameter high pressure polyethylene tubing used for the conduit sections 62, 63, 64 and 66, for example, with each fitting 60 being disposed in a void or space, such as the spaces 48. One source of spray nozzle commercially available is of a type used for water misting systems for horticultural or human comfort purposes in out of doors facilities. Equipment available from Arizona Mist of Gilbert Ariz. may be suitable for the system 45 described and shown. As previously mentioned, systems in accordance with the invention and, as shown in FIGS. 2 and 3, may be installed during new construction or during remodeling or rehabilitation of a building. Suitable notches or holes 42a, FIG. 2, may be formed in the column or stud members 42 for extending the conduits 62, 63 and 64 therethrough, for example. The manifolded spray nozzles 58 of system 45 are advantageously supported by the column members 42 within the spaces 48.

After installation of the system 45, for example, and upon completion of construction, the spaces 48 may be treated at any time that mold growth may occur or be anticipated. Moreover, during construction or remodeling of a building, such as the dwelling unit 10, a method in accordance with the present invention also contemplates the steps of cleanup of all construction debris which might interfere with the application of fungicide to be sprayed on a surface, such as the column members 42, the floor plate 44, the floor surface 24a, the interior wall panels such as the panels 50 and 52 and the wall panels which form the interior walls 28 and 31. All debris collected by sweeping, picking up or vacuuming the surfaces in question should, of course, be subjected to suitable disposal. Once the interior structural features of the dwelling unit are cleaned, a portable application unit including components such as the pump unit 75 and reservoir 80, may be brought to the dwelling unit and connected to a suitable portable high pressure spray nozzle, not shown, which may be manually operated.

All surfaces which are subject to mold growth should then be sprayed, including all wood and other structural features which include cellulose material, as well as concrete surfaces. For example, the subfloors 92 and 94 should be treated and the point of penetration 69 of the vent conduit 98 at the roof 71 should be sprayed. The treatment fluid is applied via the aforementioned equipment so that a predetermined fungicide particle size of from about eighteen to one hundred microns may be sprayed on the surfaces.

In addition to treating the exposed interior surfaces mentioned above, the interior surfaces of exterior walls 12, 14, 16, 18, 20 and 22 should be treated. In addition, any space which occupies water piping or structural features which are subject to accumulation of leaking water or condensed water vapor should be sprayed during the mold prevention or eradication treatment process. As described in detail above, all interior wall structural components, such as studs or columns, floor plates, and the interior of wall paneling should be sprayed as well as subfloors which support equipment which is likely to leak water or to shed condensed water vapor. Still further, the perimeter of all floors from the outer edge thereof to at least about two feet toward the interior of the structure should be sprayed with fungicide prior to the application of floor coverings. As shown in FIG. 1, the floor surface 24a should be treated inward from the exterior walls 12, 14, 16, 18, 20, and 22 a distance of up to about two feet as indicated by the treatment lines 12a and 14a in FIG. 1, for example.

The above-described methods and system are exemplary. Various residential dwelling structures and commercial buildings will have other structural features which will advantageously utilize the treatment system and methods in accordance with the invention. For example, in many multi-story buildings or buildings with basements, the interior floors may be other than concrete slabs and be built up of spaced apart structural members with subflooring disposed thereover and paneling disposed thereunder. The interior spaces formed by such flooring structures are also susceptible, in many instances, to mold growth and should have installed therein a system as described hereinabove and shown by way of example in FIGS. 2 and 3.

Although preferred embodiments of a system and methods for mold prevention and eradication have been described in detailed herein, those skilled in the art will also recognize that various substitutions and modifications may be made without departing from the scope and spirit of the appended claims.

What is claimed is:

1. A system for the prevention or eradication of vermin, including fungi, in a residential or commercial building, characterized by:
at least one spray nozzle mounted in a hidden void or space within said building susceptible to moisture accumulation, a conduit connected to said spray nozzle and to a connector for connecting said spray nozzle to a source of pressure fluid to be injected into said void or space to prevent or eradicate growth of said fungi.

2. The system set forth in claim 1 wherein:
said building includes at least one interior wall including spaced apart column members and means forming a wall panel on opposite sides of said column members and defining plural spaces, and said system includes a spray nozzle mounted in selected ones of said spaces, said spray nozzles being interconnected by conduit means and operably connected to said connector for receiving high pressure fluid for delivery into said spaces, respectively.

3. The system set forth in claim 2 wherein:
said spray nozzles are operable to deliver liquid treatment fluid in droplets having a particle size of from about 18 to 100 microns.

4. The system set forth in claim 2 wherein:
said spray nozzles are disposed in spaces occupied by pressure water conduits.

5. The system set forth in claim 2 wherein:
said connector includes a connector part projecting through one of said wall panels at a point normally hidden from view of an occupant of said building.

6. The system set forth in claim 2 wherein:
said spray nozzles and said conduit means are supported by said column members.

7. A system for the prevention or eradication of vermin, including fungi, in a residential or commercial building, wherein said building includes at least one interior wall including spaced apart column members and means forming a wall panel on opposite sides of said column members and defining plural spaces, said system including:
spray nozzles mounted in selected ones of said spaces, said spray nozzles being interconnected by conduit means and operably connected to a connector for receiving high pressure fungicide for delivery into said spaces, respectively, and said connector includes a connector part projecting through one of said wall panels at a point normally hidden from view of an occupant of said building.

8. The system set forth in claim 7 wherein:
said spray nozzles are operable to deliver liquid fungicide in droplets having a particle size of from about 18 to 100 microns.

9. A method for preventing or eliminating the growth of mold in interior spaces of a building to be occupied by human beings, said building including at least one interior space occupied by a source of moisture sufficient to promote the growth of mold on surfaces of said building, comprising the steps of:
installing at least one spray nozzle within said space, said spray nozzle being connected to a conduit and to a connector for said conduit for conducting high pressure liquid treatment fluid to said spray nozzle;
enclosing said space to complete construction or renovation of said building; and
from time to time connecting a source of pressure liquid to said connector for injecting a spray of liquid into said space.

10. The method set forth in claim 9, including the step of:
installing said spray nozzle between two spaced apart column members of an interior wall of said building for spraying the surfaces of said column members and opposed wall panels forming said wall with said treatment fluid.

11. The method set forth in claim 10, including the steps of:
installing plural spray nozzles in spaced apart spaces formed in said interior wall, interconnecting said spray nozzles with conduit means and connecting said connector to said spray nozzles with further conduit means for receiving pressure liquid from time to time.

12. The method set forth in claim 11, including the step of:
supporting said spray nozzles and said conduit means within said spaces by said column members.

13. The method set forth in claim 9, including the steps of:
installing said system within plural interior walls of said building, said interior walls including sources of moisture accumulation.

14. The method set forth in claim 9, including the step of:
applying a spray of liquid treatment fluid to exposed interior surfaces of selected exterior walls of said building.

15. The method set forth in claim 9, including the step of:
applying a spray of liquid treatment fluid to plumbing chase spaces of said building.

16. The method set forth in claim 9, including the steps of:
applying a spray of liquid treatment fluid to column and floor plate members defining walls of said building.

17. The method set forth in claim 9, including the step of:
applying a spray of liquid treatment fluid to floor surfaces in all spaces having a source of water flooding selected from a group consisting of subfloors for utility appliances, interior wood floors and concrete floors.

18. A method for preventing or eliminating the growth of mold in interior spaces of a building to be occupied by human beings, said building including interior spaces occupied by a source of moisture sufficient to promote the growth of mold on surfaces of said building, comprising the steps of:
applying a spray of liquid anti-microbial treatment fluid to plumbing chase spaces of said building;
applying a spray of said fluid to column and floor plate members defining walls of said building; and
applying a spray of said fluid to floor surfaces in spaces having a source of water flooding selected from a group consisting of subfloors for utility appliances, interior wood floors and concrete floors.

19. The method set forth in claim 18 including the step of:
applying a spray of said fluid to exposed interior surfaces of at least selected ones of exterior walls of said building.

20. The method set forth in claim 18 including the step of:
applying a spray of said fluid to roof penetrations of said building.

21. The method set forth in claim 18 including the step of:
applying a spray of said fluid to the interior surfaces of floors of said building within an area extending from exterior walls of said building inward a predetermined distance.

22. The method set forth in claim 18 including the step of:
removing all construction debris interfering with the application of said fluid prior to applying a spray of said fluid.

23. The method set forth in claim 18 including the step of:
installing plural spray nozzles in spaced apart spaces formed in interior walls of said building and interconnecting said spray nozzles with conduit means including a connector for receiving pressure liquid treatment fluid from time to time.
